(19) United States
(12) Patent Application Publication
(10) Pub. No.: US 2009/0166015 A1
(43) Pub. Date: Jul. 2, 2009

(54) Heat Sink and Electronic Device with Fragrance

(76) Inventors: Tzu-Wei Lin, Taipei City (TW); Chen-Ming Sun, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/246,572

(22) Filed: Oct. 7, 2008

(30) Foreign Application Priority Data
Dec. 27, 2007 (TW) 096150524

(51) Int. Cl. F28F 13/18; A61L 9/03

(52) U.S. Cl. 165/133; 422/125

(57) ABSTRACT

A heat sink with fragrance is disclosed. The heat sink comprises a heat-dissipating unit and fragrant coating materials, wherein the fragrant coating materials are applied on at least a portion of the heat-dissipating unit; the fragrant coating materials comprise fragrant materials capable of releasing fragrance; and a heat generation element contacts the heat-dissipating unit.

### BACKGROUND OF THE INVENTION

1. Field of the Invention

The present invention relates to a heat sink and electronic device; more particularly, the present invention relates to a heat sink and electronic device with fragrance.

2. Description of the Related Art

For a general electronic device (such as a laptop computer or a mobile phone), in addition to functionality requirements, most manufacturers emphasize the design of its appearance. However, some senses other than sight, such as touch and smell, have become popular in recent years.

In prior arts, some manufacturers mix fragrant materials with rubber or silicone materials and apply injection-molding techniques to make products with fragrance (such as toys). However, because it is not easy for fragrant materials to be mixed with rubber or silicone materials and thereby form a solid shape, the application of such kind of products is somewhat limited.

Therefore, there is a need to provide a heat sink and electronic device with fragrance to mitigate and/or obviate the aforementioned problems.

### SUMMARY OF THE INVENTION

It is an object of the present invention to provide a heat sink with fragrance.

It is another object of the present invention to provide an electronic device having a heat sink with fragrance.

To achieve the aforementioned objects, the heat sink with fragrance of the present invention comprises a heat-dissipating unit and fragrant coating materials. The fragrant coating materials are applied on at least a portion of the heat-dissipating unit. The fragrant coating materials comprise fragrant materials capable of releasing fragrance. In addition, there is a heat generation element contacting the heat-dissipating unit.

To achieve another aforementioned object, the electronic device of the present invention comprises a heat generation element and a heat sink with fragrance. The heat sink with fragrance comprises a heat-dissipating unit and fragrant coating materials. The fragrant coating materials are applied on at least a portion of the heat-dissipating unit. The fragrant coating materials comprise fragrant materials capable of releasing fragrance. The heat generation element contacts the heat-dissipating unit.

Other objects, advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1 is a schematic drawing of an electronic device of the present invention.

FIG. 2 is a schematic drawing of a heat sink of the present invention.

FIG. 3 illustrates an A-A cross-sectional view of the heat sink as shown in FIG. 2 of the present invention.

### DETAILED DESCRIPTION OF THE PREFERRED EMBODIMENT

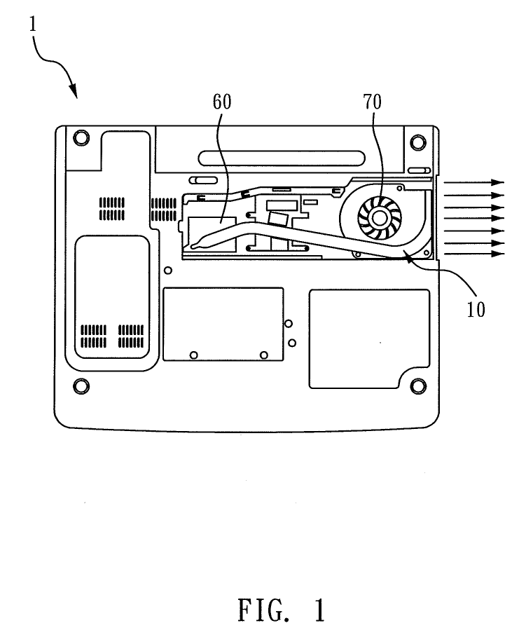

Please refer to FIG. 1. FIG. 1 is a schematic drawing of an electronic device of the present invention. The electronic device 1 comprises a heat sink 10 with fragrance. The heat sink 10 in this embodiment is a general device for achieving a heat dissipation object. The heat sink 10 is connected to a heat generation element 60.

The heat generation element 60 in this embodiment represents any kinds of elements capable of generating heat when the electronic device 1 is working. For example, the heat generation element 60 can be a central processing unit (CPU). The heat generation element 60 can directly contact a heat-dissipating unit 20 or fragrant coating materials 30, such that the heat generated by the heat generation element 60 can be transmitted via the heat sink 10. Forced convection generated by a fan 70 can speed up the process of spreading the heat of the heat-dissipating unit 20, thereby increasing the heat dissipation efficiency of the heat generation element 60.

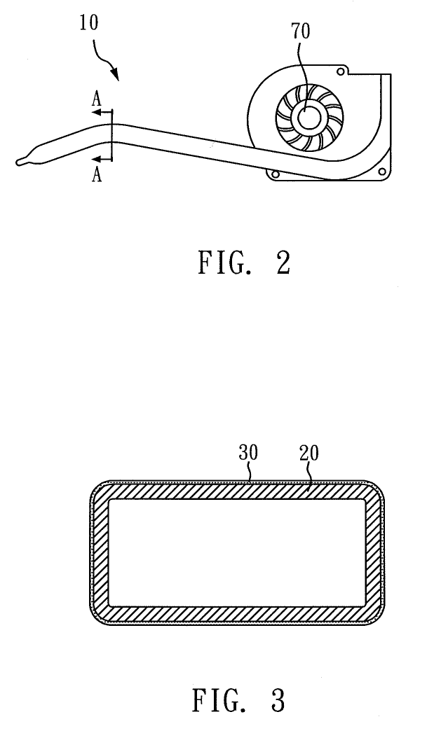

Please refer to both FIG. 2 and FIG. 3. FIG. 2 is a schematic drawing of a heat sink of the present invention. FIG. 3 illustrates an A-A cross-sectional view of the heat sink as shown in FIG. 2 of the present invention.

In this embodiment, the heat sink 10 comprises a heat-dissipating unit 20, fragrant coating materials 30, and a fan 70. Please note that the structure of the heat sink is not limited to this embodiment. For example, when the heat sink utilizes natural convection to perform a heat dissipation process, the heat sink does not have to include the fan.

The heat-dissipating unit 20 is an element with good thermal conduction so as to rapidly spread the heat generated by the heat generation element 60. In this embodiment, the heat-dissipating unit 20 is a hollow heat pipe. Please note that the heat-dissipating unit can also be a fin or any other equivalent element with a heat dissipation function.

The fragrant coating materials 30 can release fragrance. In this embodiment, the fragrant coating materials 30 comprise oil and essence, wherein the purpose of oil is to increase the adhesion, such that the fragrance of the fragrant coating materials 30 can last longer. Please note that the fragrant coating materials do not have to include oil.

The essence can be in the form of a powder (such as an essence powder composed of microencapsulated capsules) or a liquid. In addition, the essence can release a plant fragrance (such as lavender fragrance or tulip fragrance) or a certain fragrance representing a brand image. Please note that the fragrance which the fragrant coating materials 30 can release is not limited to this embodiment.

The fragrant coating materials 30 are applied on at least a portion of the heat-dissipating unit 20. In this embodiment, the fragrant coating materials 30 are applied on the outside of the heat-dissipating unit 20 such that the fragrant coating materials 30 can release fragrance by means of the heat generated by the heat generation element 60. Please note that the location of the heat-dissipating unit 20 on which the fragrant coating materials 30 are applied is not limited to this embodiment.

The following description explains the operation of the electronic device 1 in detail. In this embodiment, when the electronic device 1 is working, the temperature of the heat generation element 60 increases, and the heat thus generated by the heat generation element 60 is transmitted into the heat-dissipating unit 20 contacting the heat generation element 60. At this time, the fragrant coating materials 30 applied on the heat-dissipating unit 20 release more fragrance because of the increased temperature. Then the fragrance is transmitted to the outside of the electronic device 1 via the fan 70 such that a user can smell the fragrance.

When the fragrant coating materials 30 are applied on only one portion of the heat-dissipating unit 20 (for example, the fragrant coating materials 30 are not applied on a portion of the heat-dissipating unit 20 near the heat generation element 60), the heat generation element 60 can directly contact the heat-dissipating unit 20. When the fragrant coating materials 30 are applied on all outside surfaces of the heat-dissipating unit 20, the heat generation element 60 directly contacts the fragrant coating materials 30. Please note that the heat sink 10 does not have to directly contact the heat generation element 60.

Although the present invention has been explained in relation to its preferred embodiments, it is to be understood that many other possible modifications and variations can be made without departing from the spirit and scope of the invention as hereinafter claimed.

What is claimed is:

1. A heat sink with fragrance, applied in an electronic device, the heat sink comprising:
a heat-dissipating unit, installed in the electronic device; and
fragrant coating materials, applied on at least a portion of the heat-dissipating unit, the fragrant coating materials being able to release fragrance.

2. The heat sink as claimed in claim 1, wherein the heat-dissipating unit is a heat pipe or a fin.

3. The heat sink as claimed in claim 1, wherein the fragrant coating materials comprise oil.

4. The heat sink as claimed in claim 1, wherein the fragrant coating materials comprise essence.

5. The heat sink as claimed in claim 1, wherein the fragrant coating materials comprise oil and essence.

6. The heat sink as claimed in claim 5, wherein the essence is in the form of a powder or a liquid.

7. The heat sink as claimed in claim 1, wherein the fragrant coating materials are applied on the outside of the heat-dissipating unit, such that the fragrant coating materials can release the fragrance by means of the heat generated by the electronic device.

8. The heat sink as claimed in claim 1, the electronic device comprising a heat generation element, wherein the heat generation element is a processor.

9. The heat sink as claimed in claim 1, wherein the fragrant coating materials release a plant fragrance.

10. An electronic device, comprising:
a heat generation element; and
a heat sink with fragrance, the heat sink comprising:
a heat-dissipating unit, disposed on the heat generation element; and
fragrant coating materials, applied on at least a portion of the heat-dissipating unit, the fragrant coating materials being able to release fragrance.

11. The electronic device as claimed in claim 10, wherein the heat-dissipating unit is a heat pipe or a fin.

12. The electronic device as claimed in claim 10, wherein the fragrant coating materials comprise oil.

13. The electronic device as claimed in claim 10, wherein the fragrant coating materials comprise essence.

14. The electronic device as claimed in claim 10, wherein the fragrant coating materials comprise oil and essence.

15. The electronic device as claimed in claim 14, wherein the essence is in the form of a powder or a liquid.

16. The electronic device as claimed in claim 10, wherein the fragrant coating materials are applied on the outside of the heat-dissipating unit, such that the fragrant coating materials can release fragrance by means of the heat generated by the electronic device.

17. The electronic device as claimed in claim 10, wherein the heat generation element is a processor.

18. The electronic device as claimed in claim 10, wherein the fragrant coating materials release a plant fragrance.
